(19) United States
(12) Patent Application Publication
(10) Pub. No.: US 2012/0149077 A1
(43) Pub. Date: Jun. 14, 2012

(54) Mesophilic and Thermophilic Organisms Modified to Produce Acrylate, and Methods of Use Thereof

(75) Inventors: Arthur J. Shaw, IV, Grantham, NH (US); Vineet Rajgarhia, Dublin, CA (US)

(73) Assignee: Mascoma Corporation, Lebanon, NH (US)

(21) Appl. No.: 13/256,297

(22) PCT Filed: Mar. 12, 2010
(86) PCT No.: PCT/US2010/027190

§ 371 Date: Feb. 27, 2012

Related U.S. Application Data
(60) Provisional application No. 61/160,125, filed on Mar. 13, 2009.

(51) Int. Cl. C12P 7/40; C12N 15/74; C12N 1/21

(52) U.S. Cl. 435/136; 435/252.3; 435/252.31; 435/320.1

(57) ABSTRACT

The present invention provides for novel metabolic pathways leading to acrylate formation in a consolidated bio-processing system (CBP) where lignocellulosic biomass is efficiently converted to acrylate. In one such metabolic pathway, pyruvate is converted to lactate, which is converted to lactoyol-CoA, which is converted to acryloyl-CoA, and which is finally converted to acrylate. In another such metabolic pathway, pyruvate is converted to L-α-alanine, which is converted to L-aspartate, which is converted to β-alanine, which is converted to β-alanyl-CoA, which is converted to acryloyl-CoA, and which is finally converted to acrylate. In yet another metabolic pathway, pyruvate is converted to lactate, and then lactate is converted directly to acrylate. In certain aspects, the invention provides for heterologous expression of one or more enzymes in a mesophilic or thermophilic organism, such as Thermoanaerobacterium saccharolyticum or Clostridium thermocellutn, where the one or more enzymes functions within a novel metabolic pathway as described above to convert pyruvate to acrylate via lactate, or via β alanine and acryloyl-CoA.

### BACKGROUND OF THE INVENTION

### Field of the Invention

Energy conversion, utilization and access underlie many of the great challenges of our time, including those associated with sustainability, environmental quality, security, and poverty. New applications of emerging technologies are required to respond to these challenges. Biotechnology, one of the most powerful of the emerging technologies, can give rise to important new energy conversion processes. Plant biomass and derivatives thereof are a resource for the biological conversion of energy to forms useful to humanity.

Among forms of plant biomass, lignocellulosic biomass (“biomass”) is particularly well-suited for energy applications because of its large-scale availability, low cost, and environmentally benign production. In particular, many energy production and utilization cycles based on cellulosic biomass have near-zero greenhouse gas emissions on a life-cycle basis. The primary obstacle impeding the more widespread production of energy from biomass feedstocks is the general absence of low-cost technology for overcoming the recalcitrance of these materials to conversion into useful products. Lignocellulosic biomass contains carbohydrate fractions (e.g., cellulose and hemicellulose) that can be converted into ethanol or other products such as lactic acid. In order to convert these fractions, the cellulose and hemicellulose must ultimately be converted or hydrolyzed into monosaccharides; it is the hydrolysis that has historically proven to be problematic.

Biologically mediated processes are promising for energy conversion. Biomass processing schemes involving enzymatic or microbial hydrolysis commonly involve four biologically mediated transformations: (1) the production of saccharolytic enzymes (cellulases and hemicellulases); (2) the hydrolysis of carbohydrate components present in pretreated biomass to sugars; (3) the fermentation of hexose sugars (e.g., glucose, mannose, and galactose); and (4) the fermentation of pentose sugars (e.g., xylose and arabinose). These four transformations occur in a single step in a process configuration called consolidated bioprocessing (CBP), which is distinguished from other less highly integrated configurations in that it does not involve a dedicated process step for cellulase and/or hemicellulase production.

CBP offers the potential for lower cost and higher efficiency than processes featuring dedicated cellulase production. The benefits result in part from avoided capital costs, substrate and other raw materials, and utilities associated with cellulase production. In addition, several factors support the realization of higher rates of hydrolysis, and hence reduced reactor volume and capital investment using CBP, including enzyme-microbe synergy and the use of thermophilic organisms and/or complexed cellulase systems. Moreover, cellulose-adherent cellulolytic microorganisms are likely to compete successfully for products of cellulose hydrolysis with non-adhered microbes, e.g., contaminants, which could increase the stability of industrial processes based on microbial cellulose utilization. Progress in developing CBP-enabling microorganisms is being made through two strategies: engineering naturally occurring cellulolytic microorganisms to improve product-related properties, such as yield and titer; and engineering non-cellulolytic organisms that exhibit high product yields and titers to express a heterologous cellulase and hemicellulase system enabling cellulose and hemicellulose utilization.

Many bacteria have the ability to ferment simple hexose sugars into a mixture of acidic and pH-neutral products via the process of glycolysis. The glycolytic pathway is abundant and comprises a series of enzymatic steps whereby a six carbon glucose molecule is broken down, via multiple intermediates, into two molecules of the three carbon compound pyruvate. This process results in the net generation of ATP (biological energy supply) and the reduced cofactor NADH.

Pyruvate is an important intermediary compound of metabolism. For example, under aerobic conditions pyruvate can be oxidized to acetyl coenzyme A (acetyl CoA), which then enters the tricarboxylic acid cycle (TCA), which in turn generates synthetic precursors, CO₂ and reduced cofactors. The cofactors are then oxidized by donating hydrogen equivalents, via a series of enzymatic steps, to oxygen resulting in the formation of water and ATP. This process of energy formation is known as oxidative phosphorylation.

Under anaerobic conditions (no available oxygen), fermentation occurs in which the degradation products of organic compounds serve as hydrogen donors and acceptors. Excess NADH from glycolysis is oxidized in reactions involving the reduction of organic substrates to products, such as lactate and ethanol. In addition, ATP is regenerated from the production of organic acids in a process known as substrate level phosphorylation. Therefore, the fermentation products of glycolysis and pyruvate metabolism include a variety of organic acids, alcohols and CO₂.

Most facultative anaerobes metabolize pyruvate aerobically via pyruvate dehydrogenase (PDH) and the tricarboxylic acid cycle (TCA). Under anaerobic conditions, the main energy pathway for the metabolism of pyruvate is via pyruvate-formate-lyase (PFL) pathway to give formate and acetyl-CoA. Acetyl-CoA is then converted to acetate, via phosphotransacetylase (PTA) and acetate kinase (ACK) with the co-production of ATP, or reduced to ethanol via acetalaldehyde dehydrogenase (AcDH) and alcohol dehydrogenase (ADH). In order to maintain a balance of reducing equivalents, excess NADH produced from glycolysis is re-oxidized to NAD⁺ by lactate dehydrogenase (LDH) during the reduction of pyruvate to lactate. NADH can also be re-oxidized by AcDH and ADH during the reduction of acetyl-CoA to ethanol, but this is a minor reaction in cells with a functional LDH. A simplified version of the central metabolic pathway leading to mixed fermentation end products in cellulolytic clostridia is presented in FIG. 2.

While the production of lactate and ethanol is observed via metabolism in various facultative anaerobes, acrylate (acrylic acid, 2-propenoic acid, prop-2-enoic acid) is rarely seen as an end product in microbial metabolism, particularly of anaerobic fermentation. More common than the production of acrylate is the production of the metabolic intermediate acryloyl-CoA, which occurs prominently in the metabolism of the propionic acid producing bacterium Clostridium propionicum (C. propionicum) and a small group of other known prokaryotic species using a similar fermentation pathway. The highest concentration of observed acrylate produced from alanine in native clostridial species is not higher than 0.2 g/L without the addition of special chemical electron acceptors.

Acrylate is a high value chemical intermediate used commercially for the synthesis of polymers. The polymers made of acrylate and its derivatives are characterized by colorless transparency, easy adhesion, elasticity, and stability to light, moderate heat as well as weathering. Acrylate is widely applied in the synthesis of highly absorbent materials, surface coatings, textiles, adhesives, paper treatment, polishes, leather, fibers, and detergents. Acrylate is currently produced from petroleum based products, primarily propene. See Xiaobo et al., “Advances in the Research and Development of Acrylic Acid Production from Biomass,” Chinese J. Chem. Eng. 14: 419-426 (2006).

One method by which acrylic acid can be produced is carried out in two steps: (1) conversion of available carbohydrates to lactic acid by fermentation; and (2) dehydration of lactic acid to acrylic acid. The dehydration process of lactic acid, however, is inefficient, generating low yields of acrylic acid. Alternative methods for increasing production of acrylate, such as via metabolic engineering of microorganisms, have been suggested, but to date no studies have yet reported the efficient production of acrylate via an engineered organism.

There is therefore a need in the art for the production of acrylate via an engineered organism, and in particular, a single engineered organism capable of converting cellulosic biomass into acrylate as part of a consolidated bioprocessing (CBP) system.

### BRIEF SUMMARY OF THE INVENTION

The present invention provides for novel metabolic pathways leading to acrylate formation in a consolidated bioprocessing system (CBP) where lignocellulosic biomass is efficiently converted to acrylate. In one such metabolic pathway, pyruvate is converted to lactate, which is converted to lactoyol-CoA, which is converted to acryloyl-CoA, and which is finally converted to acrylate. In another such metabolic pathway, pyruvate is converted to L-α-alanine, which is converted to L-aspartate, which is converted to β-alanine, which is converted to β-alanyl-CoA, which is converted to acryloyl-CoA, and which is finally converted to acrylate. In yet another metabolic pathway, pyruvate is converted to lactate, and then lactate is converted directly to acrylate. The novel metabolic pathways described above are set forth in FIGS. 4-6.

One aspect of the invention relates to enzymes that function within such novel metabolic pathways of the invention. In particular, the invention provides for heterologous expression of one or more of such enzymes in a mesophilic or thermophilic organism, such as Thermoanaerobacterium saccharolyticum (T. saccharolyticum) or Clostridium thermocellum (C. thermocellum).

The invention further provides for an isolated nucleic acid molecule, or a complement thereof, comprising a nucleotide sequence of an enzyme that functions within a novel metabolic pathway of the invention as described above. In certain embodiments, such an enzyme is involved in the conversion of the starting product (pyruvate) to an intermediate product, such as lactate. In further embodiments, such an enzyme is involved in the conversion of one intermediate product to another. In certain other embodiments, such an enzyme is involved in the conversion of one intermediate product to the final product (acrylate).

In certain embodiments, such an enzyme directly converts pyruvate to lactate, or directly converts lactate to acrylate, as set forth in FIG. 4. In particular aspects of the invention, such an enzyme that directly converts pyruvate to lactate, or directly converts lactate to acrylate, shares at least 70%, 75%, 80%, 85%, 90%, 95%, 97%, 98% or 100% identity to the C. propionicum enzyme D-lactate dehydrogenase (EC 1.1.1.28) or hypothetical lactate dehydratase (EC 4.2.1.x).

In certain embodiments, such an enzyme converts lactoyl-CoA to acryloyl-CoA, or converts acryloyl-CoA to acrylate, where lactoyl-CoA and acryloyl-CoA are intermediate products generated during conversion of lactate to acrylate, as set forth in FIG. 5. In particular aspects of the invention, such an enzyme that converts lactoyl-CoA to acryloyl-CoA, or converts acryloyl-CoA to acrylate shares at least 70%, 75%, 80%, 85%, 90%, 95%, 97%, 98% or 100% identity to the C. propionicum enzyme D-lactoyl-CoA dehydratase (EC 4.2.1.54) or hypothetical lactate (acrylate) CoA transferase (EC 2.8.3.x)

In certain embodiments, such an enzyme converts pyruvate to L-α-alanine, or converts L-α-alanine to L-aspartate, or converts L-aspartate to β-alanine, or converts β-alanyl-CoA to acryloyl-CoA, or converts acryloyl-CoA to acrylate, where L-α-alanine, L-aspartate, β-alanine, β-alanyl-CoA and acryloyl-CoA are intermediate products generated along a metabolic pathway during conversion of pyruvate to acrylate, as set forth in FIG. 6. In particular aspects of the invention, such an enzyme that converts pyruvate to L-α-alanine, or converts L-α-alanine to L-aspartate, or converts L-aspartate to β-alanine, or converts β-alanyl-CoA to acryloyl-CoA, or converts acryloyl-CoA to acrylate, where L-α-alanine, L-aspartate, β-alanine, β-alanyl-CoA and acryloyl-CoA shares at least 70%, 75%, 80%, 85%, 90%, 95%, 97%, 98% or 100% identity to the C. propionicum enzyme alanine aminotransferase (EC 2.6.1.2), glutamate dehydrogenase (EC 1.4.1.3), aspartate 4-decarboxylase (EC 4.1.1.11), β-alanyl-CoA:ammonia lyase (EC 4.3.1.6) or hypothetical β-alanine (acrylate) CoA transferase (EC 2.8.3.x).

The present invention is further directed to polypeptides encoded by the nucleotide sequences as described above.

A further aspect of the present invention relates to a genetic construct comprising a nucleic acid sequence of an enzyme capable of converting pyruvate to acrylate via a metabolic pathway as described above, operably linked to a promoter expressible in a thermophilic or mesophilic bacterium. In a further embodiment of the invention, the genetic construct comprising such a nucleic acid sequence is introduced and expressed in a thermophilic or mesophilic bacterium. The present invention also relates to a recombinant thermophilic or mesophilic bacterium comprising the aforementioned genetic construct.

The present invention also encompasses a vector comprising any one of the aforementioned nucleic acid molecules. The present invention also encompasses a host cell comprising any one of the aforementioned nucleotide or polypeptide sequences. In certain embodiments, the invention relates to the aforementioned host cell, wherein said host cell is a thermophilic or mesophilic bacterial cell.

Another aspect of the invention relates to a genetically modified thermophilic or mesophilic microorganism, wherein one or more native genes are partially, substantially, or completely deleted, silenced, inactivated, or down-regulated, wherein said one or more native genes encodes for one or more native enzymes involved in pathways leading to acetate, ethanol, and/or formate production, thereby increasing the native ability of said thermophilic or mesophilic microorganism to produce lactate via a competing pathway as a fermentation product. In at least two of the novel metabolic pathways of the present invention, lactate is a necessary metabolic intermediate for the production of acrylate. Thus, shifting production, for example, from acetate and ethanol towards lactate will also improve the ultimate yield of acrylate. In certain embodiments, the present invention relates to the aforementioned genetically modified microorganism, wherein said one or more native enzymes is acetate kinase (ack), phosphotransacetylase (pta), alcohol dehydrogenase (adh), and/or pyruvate-formate-lyase (O).

In certain embodiments, the present invention relates to the aforementioned genetically modified microorganism, wherein said microorganism is a Gram-negative bacterium or a Gram-positive bacterium. In certain embodiments, the present invention relates to the aforementioned genetically modified microorganism, wherein said microorganism is a species of the genera Thermoanaerobacterium, Thermoanaerobacter, Clostridium, Geobacillus, Saccharococcus, Paenibacillus, Bacillus, Caldicellulosiruptor, Anaerocellum, or Anoxybacillus. In certain embodiments, the present invention relates to the aforementioned genetically modified microorganism, wherein said microorganism is a bacterium selected from the group consisting of: Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium therm osaccharolyticum, Thermoanaerobacter thermohydrosulfuricus, Thermoanaerobacter ethanolicus, Thermoanaerobacter brocki, Clostridium thermocellum, Clostridium cellulolyticum, Clostridium phytofermentans, Clostridium straminosolvens, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, and Anaerocellum thermophilum. In certain embodiments, the present invention relates to the aforementioned genetically modified microorganism, wherein said microorganism is Clostridium thermocellum, Clostridium cellulolyticum, or Thermoanaerobacterium saccharolyticum.

In certain embodiments, the present invention relates to the aforementioned genetically modified microorganism, wherein one or more non-native (or heterologous) genes encodes for one or more non-native enzymes that function to convert pyruvate into acrylate via the novel metabolic pathways described above. In particular embodiments, the one or more non-native enzymes is an enzyme that converts pyruvate to lactate, converts lactate to acrylate, converts lactoyl-CoA to acryloyl-CoA, converts acryloyl-CoA to acrylate, converts pyruvate to L-α-alanine, converts L-α-alanine to L-aspartate, converts L-aspartate to β-alanine, converts β-alanyl-CoA to acryloyl-CoA, or converts acryloyl-CoA to acrylate.

In further embodiments, such one or more non-native enzymes is from a mesophilic or thermophic organism, such as C. propionicum. Examples of enzymes from C. propionicum include D-lactate dehydrogenase (EC 1.1.1.28), hypothetical lactate dehydratase (EC 4.2.1.x), D-lactoyl-CoA dehydratase (EC 4.2.1.54), hypothetical lactate (acrylate) CoA transferase (EC 2.8.3.x), alanine aminotransferase (EC 2.6.1.2), glutamate dehydrogenase (EC 1.4.1.3), aspartate 4-decarboxylase (EC 4.1.1.11), β-alanyl-CoA:ammonia lyase (EC 4.3.1.6) and hypothetical β-alanine (acrylate) CoA transferase (EC 2.8.3.x). In additional embodiments, the one or more non-native enzymes is an enzyme homologous to a C. propionicum enzyme that converts pyruvate to lactate, converts lactate to acrylate, converts lactoyl-CoA to acryloyl-CoA, converts acryloyl-CoA to acrylate, converts pyruvate to L-α-alanine, converts L-α-alanine to L-aspartate, converts L-aspartate to β-alanine, converts β-alanyl-CoA to acryloyl-CoA, or converts acryloyl-CoA to acrylate.

In other embodiments, the one or more non-native (or heterologous) genes encodes one or more non-native enzymes that confers the ability to metabolize a hexose sugar or pentose sugar, thereby allowing said thermophilic or mesophilic microorganism to increase production of lactate as a fermentation product from a hexose sugar or a pentose sugar, and ultimately thereby allowing the increased production of acrylate. In certain embodiments, the present invention relates to the aforementioned genetically modified microorganism, wherein said one or more non-native enzymes is lactate dehydrogenase (ldh).

Yet another aspect of the invention relates to a genetically modified thermophilic or mesophilic microorganism, wherein (a) one or more native genes is partially, substantially, or completely deleted, silenced, inactivated, or down-regulated, which one or more native genes encodes one or more native enzymes involved in the metabolic production of an organic acid or a salt thereof, and (b) one or more non-native genes is inserted, which one or more non-native genes encodes one or more non-native enzymes involved in the metabolic production of acrylate, thereby allowing said thermophilic or mesophilic microorganism to produce acrylate as a fermentation product.

In certain embodiments, the present invention relates to any of the aforementioned genetically modified microorganisms, wherein said microorganism is mesophilic. In certain embodiments, the present invention relates to any of the aforementioned genetically modified microorganisms, wherein said microorganism is thermophilic.

Another aspect of the invention relates to a process for converting lignocellulosic biomass to acrylate, comprising contacting lignocellulosic biomass with any one of the aforementioned genetically modified thermophilic or mesophilic microorganisms. In certain embodiments, the present invention relates to the aforementioned process, wherein said lignocellulosic biomass is selected from the group consisting of grass, switch grass, cord grass, rye grass, reed canary grass, mixed prairie grass, miscanthus, sugar-processing residues, sugarcane bagasse, sugarcane straw, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, and combinations thereof. In certain embodiments, the present invention relates to the aforementioned process, wherein said lignocellulosic biomass is selected from the group consisting of corn stover, sugarcane bagasse, switchgrass, and poplar wood. In certain embodiments, the present invention relates to the aforementioned process, wherein said lignocellulosic biomass is corn stover. In certain embodiments, the present invention relates to the aforementioned process, wherein said lignocellulosic biomass is sugarcane bagasse. In certain embodiments, the present invention relates to the aforementioned process, wherein said lignocellulosic biomass is switchgrass. In certain embodiments, the present invention relates to the aforementioned process, wherein said lignocellulosic biomass is poplar wood. In certain embodiments, the present invention relates to the aforementioned process, wherein said lignocellulosic biomass is willow. In certain embodiments, the present invention relates to the aforementioned process, wherein said lignocellulosic biomass is paper sludge.

### BRIEF DESCRIPTION OF THE DRAWINGS/FIGURES

FIG. 1 depicts the glycolysis pathway.

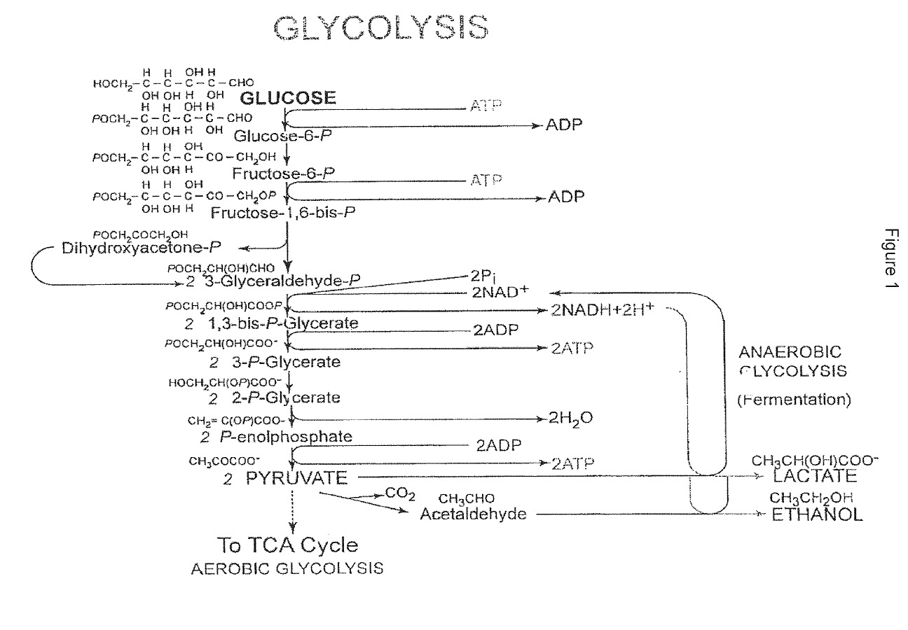

FIG. 2 shows a schematic of a simplified version of central metabolic pathways leading to mixed acid fermentation end products of cellulolytic clostridia.

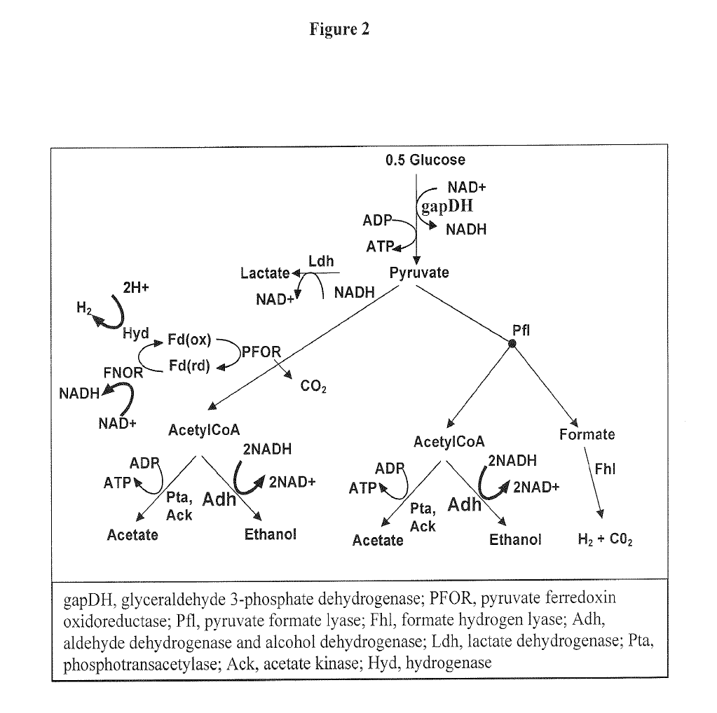

FIG. 3 shows a schematic of the metabolic pathway of alanine fermentation from C. propionicum.

FIG. 4 shows a schematic of a theoretical pathway for conversion of pyruvate to acrylate where pyruvate is converted to lactate, and then lactate is converted directly to acrylate. The theoretical pathway has been designed by combining metabolic steps that occur in the native T. saccharolyticum or C. thermocellum organisms (solid black line), in the native C. propionicum organism (solid grey line), or are theoretical metabolic steps (dotted line).

FIG. 5 shows a schematic of a theoretical pathway for conversion of pyruvate to acrylate, where pyruvate is converted to lactate, which is converted to lactoyol-CoA, which is converted to acryloyl-CoA, and which is finally converted to acrylate. The solid black line, solid grey line and dotted line represent metabolic step as described above for FIG. 4.

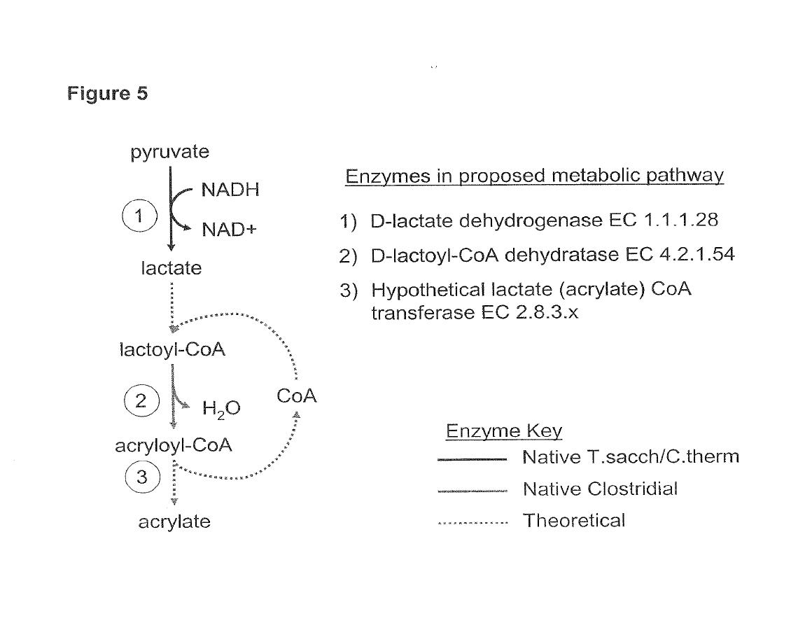

FIG. 6 shows a schematic of a theoretical pathway for conversion of pyruvate to acrylate, where pyruvate is converted to L-α-alanine, which is converted to L-aspartate, which is converted to β-alanine, which is converted to β-alanyl-CoA, which is converted to acryloyl-CoA, and which is finally converted to acrylate. The solid black line, solid grey line and dotted line represent metabolic step as described above for FIG. 4.

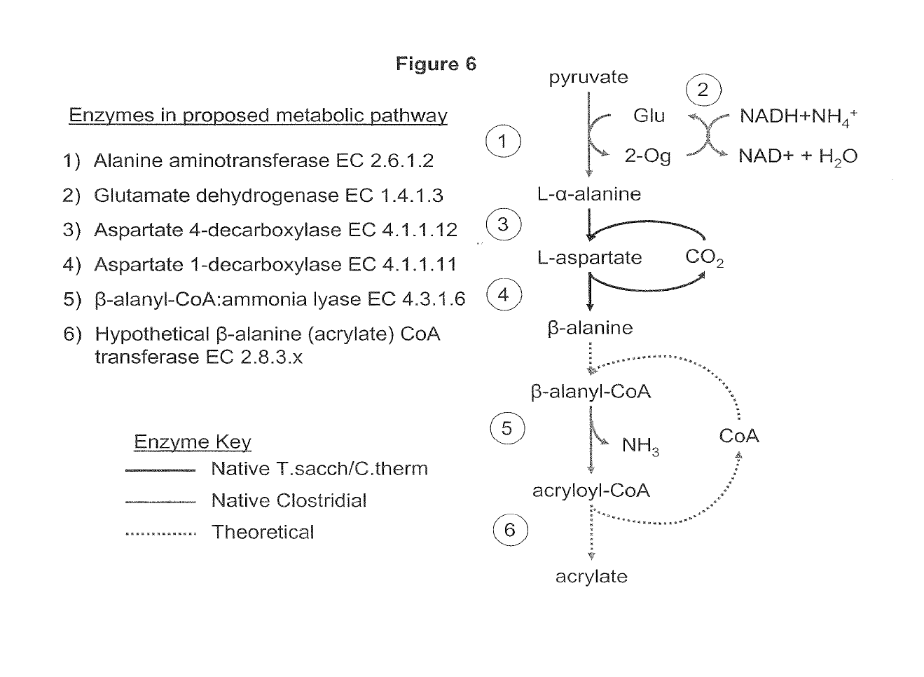

### DETAILED DESCRIPTION OF THE INVENTION

Aspects of the present invention relate to the engineering of thermophilic or mesophilic microorganisms for use in the production of acrylate from lignocellulosic biomass. The use of thermophilic bacteria for acrylate production offers many advantages over traditional processes based upon mesophilic ethanol producers. For example, the use of thermophilic organisms provides significant economic savings over traditional process methods due to lower acrylate separation costs, reduced requirements for external enzyme addition, and reduced processing times.

Aspects of the present invention relate to a process by which the cost of acrylate production from cellulosic biomass-containing materials can be reduced by using a novel processing configuration. In particular, the present invention provides novel metabolic pathways for generating acrylate production in a genetically modified microorganism using a consolidated bioprocessing system (CBP). The novel metabolic pathways have been designed by combining metabolic steps that occur in the native T. saccharolyticum or C. thermocellum organisms with metabolic steps that occur in the native C. propionicum organism, and can include additional theoretical steps that aid in the conversion of pyruvate to acrylate.

In one such metabolic pathway, pyruvate is converted to lactate, and then lactate is converted directly to acrylate. In another such metabolic pathway, pyruvate is converted to lactate, which is converted to lactoyol-CoA, which is converted to acryloyl-CoA, and which is finally converted to acrylate. In yet another metabolic pathway, pyruvate is converted to L-α-alanine, which is converted to L-aspartate, which is converted to β-alanine, which is converted to β-alanyl-CoA, which is converted to acryloyl-CoA, and which is finally converted to acrylate.

In certain other embodiments, the present invention relates to genetically modified thermophilic or mesophilic microorganisms, wherein a gene or a particular polynucleotide sequence is partially, substantially, or completely deleted, silenced, inactivated, or down-regulated, which gene or polynucleotide sequence encodes for an enzyme that confers upon the microorganism the ability to produce lactate as a major fermentation product. Further, by virtue of a novel integration of processing steps, commonly known as consolidated bioprocessing, aspects of the present invention provide for more efficient conversion of lactate to acrylate from cellulosic-biomass-containing raw materials. The incorporation of genetically modified thermophilic or mesophilic microorganisms in the processing of said materials allows for fermentation steps to be conducted at higher temperatures, improving process economics. For example, reaction kinetics are typically proportional to temperature, so higher temperatures are generally associated with increases in the overall rate of production. Additionally, higher temperature facilitates the removal of volatile products from the broth and reduces the need for cooling after pretreatment.

In certain embodiments, the present invention relates to genetically modified or recombinant thermophilic or mesophilic microorganisms with increased ability to produce enzymes that confer the ability to convert lactate to acrylate. In one aspect of the invention, one or more non-native (or heterologous) genes are inserted into a genetically modified thermophilic or mesophilic microorganism, wherein said non-native gene encodes an enzyme involved in the metabolic conversion of pyruvate to acrylate. For example, in certain embodiments, the enzyme can be an enzyme that converts pyruvate to lactate, converts lactate to acrylate, converts lactoyl-CoA to acryloyl-CoA, converts acryloyl-CoA to acrylate, converts pyruvate to L-α-alanine, converts L-α-alanine to L-aspartate, converts L-aspartate to β-alanine, converts β-alanyl-CoA to acryloyl-CoA, or converts acryloyl-CoA to acrylate. By inserting (e.g., introducing or adding) a non-native (or heterologous) gene that encodes an enzyme involved in the conversion of pyruvate to acrylate, the microorganism has an increased ability to produce acrylate relative to the native organism.

The present invention also provides novel compositions that can be integrated into the microorganisms of the invention. In one aspect, the microorganism comprises a nucleic acid molecule expressing a heterologous enzyme. In one embodiment, an isolated nucleic acid molecule of the present invention comprises a nucleotide sequence, or a portion thereof, which is at least about 50%, 54%, 55%, 60%, 62%, 65%, 70%, 75%, 78%, 80%, 85%, 86%, 87%, 88%, 89%, 90%, 91%, 92%, 93%, 94%, 95%, 96%, 97%, 98%, 99%, or more homologous to the nucleotide sequences (e.g., to the entire length of the nucleotide sequence) encoding for a C. propionicum enzyme that converts pyruvate to lactate, converts lactate to acrylate, converts lactoyl-CoA to acryloyl-CoA, converts acryloyl-CoA to acrylate, converts pyruvate to L-α-alanine, converts L-α-alanine to L-aspartate, converts L-aspartate to β-alanine, converts β-alanyl-CoA to acryloyl-CoA, or converts acryloyl-CoA to acrylate. In particular, such an enzyme can correspond to a C. propionicum D-lactate dehydrogenase (EC 1.1.1.28), hypothetical lactate dehydratase (EC 4.2.1.x), D-lactoyl-CoA dehydratase (EC 4.2.1.54), hypothetical lactate (acrylate) CoA transferase (EC 2.8.3.x), alanine aminotransferase (EC 2.6.1.2), glutamate dehydrogenase (EC 1.4.1.3), aspartate 4-decarboxylase (EC 4.1.1.11), β-alanyl-CoA:ammonia lyase (EC 4.3.1.6) or hypothetical β-alanine (acrylate) CoA transferase (EC 2.8.3.x).

In certain embodiments, the C. propionicum enzyme corresponds to a β-alanyl-CoA:ammonia lyase 1 enzyme with GenBank Accession No. AJ715481 having the following sequence:

(SEQ ID NO: 1)
MVGKKVVHHLMMSAKDAHYTGNLVNGARIVNQWGDVGTELMVYVDGDIS
LFLGYKDIEFTAPVYVGDFMEYHGWIEKVGNQSYTCKFEAWKVATMVDI
TNPQDTRATACEPPVLCGRATGSLFIAKKDQRGPQESSFKERKHPGE.

In certain embodiments, the C. propionicum enzyme corresponds to the 3-alanyl-CoA:ammonia lyase 2 with GenBank Accession No. AJ715482 having the following sequence:

(SEQ ID NO: 2)
MVGKKVVHHLMMSAKDAHYTGNLVNGARIVNQWGDVGTELMVYVDGDIS
LFLGYKDIEFTAPVYVGDFMEYHGWIEKVGNQSYTCKFEAWKVAKMVDI
TNPQDTRATACEPPVLCGTATGSLFIAKDNQRGPQESSFKDAKHPQ.

In certain embodiments, the C. propionicum enzyme corresponds to the lactoyl-CoA dehydratase with GenBank Accession No. AJ276553 having the following sequence:

(SEQ ID NO: 3)
EFKIAIVDDDLAQESRQIRVDVLDGEGGPLYRMAKAWQQMYGCSLATDT
KKGRGRMLINKTIQTGADAIVVAMMKFCDPEEWDYPVMYREFEEKGVKS
LMIEVDQEVSSFEQIKTRLQSFVEML.

Alternatively, genes identified from mesophilic or thermophilic organisms encoding for enzymes sharing at least 70%, 75%, 80%, 85%, 90%, 95%, 97%, 98% or 100% homology with a C. propionicum enzyme that converts pyruvate to lactate, converts lactate to acrylate, converts lactoyl-CoA to acryloyl-CoA, converts acryloyl-CoA to acrylate, converts pyruvate to L-α-alanine, converts L-α-alanine to L-aspartate, converts L-aspartate to β-alanine, converts β-alanyl-CoA to acryloyl-CoA, or converts acryloyl-CoA to acrylate could be heterologously expressed in an engineered organism. For example, a gene identified from T. saccharolyticum encoding for an enzyme sharing at least 70%, 75%, 80%, 85%, 90%, 95%, 97%, 98% or 100% homology with C. propionicum that converts pyruvate to lactate, converts lactate to acrylate, converts lactoyl-CoA to acryloyl-CoA, converts acryloyl-CoA to acrylate, converts pyruvate to L-α-alanine, converts L-α-alanine to L-aspartate, converts L-aspartate to β-alanine, converts β-alanyl-CoA to acryloyl-CoA, or converts acryloyl-CoA to acrylate could be heterologously overexpressed in a mesophilic or thermophilic organism, such as T. saccharolyticum or C. thermocellum. Similarly, a gene identified from C. thermocellum encoding for an enzyme sharing at least 70%, 75%, 80%, 85%, 90%, 95%, 97%, 98% or 100% homology with a C. propionicum enzyme that converts pyruvate to lactate, converts lactate to acrylate, converts lactoyl-CoA to acryloyl-CoA, converts acryloyl-CoA to acrylate, converts pyruvate to L-α-alanine, converts L-α-alanine to L-aspartate, converts L-aspartate to β-alanine, converts β-alanyl-CoA to acryloyl-CoA, or converts acryloyl-CoA to acrylate could be heterologously overexpressed in a mesophilic or thermophilic organism, such as T. saccharolyticum or C. thermocellum.

Metabolic engineering of microorganisms for the production of acrylate could also be achieved by the heterologous expression of enzymes that function to increase production of lactate as a fermentation product from a hexose sugar or a pentose sugar, and ultimately thereby allowing the increased production of acrylate. In certain embodiments, the present invention relates to the aforementioned genetically modified microorganism, wherein said one or more non-native enzymes is lactate dehydrogenase (ldh).

In a further aspect of the invention, the microorganism comprises a polypeptide corresponding to a heterologous enzyme as described above.

Metabolic engineering of microorganisms for the production of acrylate could also be achieved by creation of a targeted knockout of the genes encoding for the production of certain enzymes, such as phosphotransacetylase or acetate kinase. In this case, “knock out” of the genes means partial, substantial, or complete deletion, silencing, inactivation, or down-regulation. If the action of lactate dehydrogenase (LDH) was maintained, and the further conversion of acetyl CoA to either acetate or ethanol was not available, i.e., if the genes encoding for the production of phosphotransacetylase (PTA) and acetate kinase (ACK) were knocked out, and/or the gene encoding for the production of ADH (alcohol dehydrogenase) was knocked out, then pyruvate could be more efficiently converted to lactate by LDH. Accordingly, a genetically modified strain of microorganism with such targeted gene knockouts, which eliminates the production of certain organic acids, would have an increased ability to produce lactate as a fermentation product, and ultimately, acrylate from lactate.

Ethanologenic organisms, such as Zymomonas mobilis, Zymobacter palmae, Acetobacter pasteurianus, or Sarcina ventriculi, and some yeasts (e.g., Saccharomyces cerevisiae), are capable of a second type of anaerobic fermentation, commonly referred to as alcoholic fermentation, in which pyruvate is metabolized to acetaldehyde and CO₂ by pyruvate decarboxylase (PDC). Acetaldehyde is then reduced to ethanol by ADH regenerating NAD⁺. Alcoholic fermentation results in the metabolism of one molecule of glucose to two molecules of ethanol and two molecules of CO₂. If the conversion of pyruvate to undesired organic acids could be avoided, as detailed above, then such a genetically modified microorganism would have an increased ability to produce lactate, and ultimately acrylate as a fermentation product.

### DEFINITIONS

The term “heterologous polynucleotide segment” is intended to include a polynucleotide segment that encodes one or more polypeptides or portions or fragments of polypeptides. A heterologous polynucleotide segment can be derived from any source, e.g., eukaryotes, prokaryotes, viruses, or synthetic polynucleotide fragments.

The terms “promoter” or “surrogate promoter” is intended to include a polynucleotide segment that can transcriptionally control a gene-of-interest that it does not transcriptionally control in nature. In certain embodiments, the transcriptional control of a surrogate promoter results in an increase in expression of the gene-of-interest. In certain embodiments, a surrogate promoter is placed 5′ to the gene-of-interest. A surrogate promoter can be used to replace the natural promoter, or can be used in addition to the natural promoter. A surrogate promoter can be endogenous with regard to the host cell in which it is used, or it can be a heterologous polynucleotide sequence introduced into the host cell, e.g., exogenous with regard to the host cell in which it is used.

The terms “gene(s)” or “polynucleotide segment” or “polynucleotide sequence(s)” are intended to include nucleic acid molecules, e.g., polynucleotides which include an open reading frame encoding a polypeptide, and can further include non-coding regulatory sequences, and introns. In addition, the terms are intended to include one or more genes that map to a functional locus. In addition, the terms are intended to include a specific gene for a selected purpose. The gene can be endogenous to the host cell or can be recombinantly introduced into the host cell, e.g., as a plasmid maintained episomally or a plasmid (or fragment thereof) that is stably integrated into the genome. In addition to the plasmid form, a gene can, for example, be in the form of linear DNA. In certain embodiments, the gene of polynucleotide segment is involved in at least one step in the bioconversion of a carbohydrate to ethanol, acetate, or lactate. Accordingly, the term is intended to include any gene encoding a polypeptide, such as the enzymes acetate kinase (ACK), phosphotransacetylase (PTA), lactate dehydrogenase (LDH), pyruvate formate lyase (PFL), aldehyde dehydrogenase (ADH) and/or alcohol dehydrogenase (ADH), enzymes for the conversion of pyruvate to acyrlate as described above. The term gene is also intended to cover all copies of a particular gene, e.g., all of the DNA sequences in a cell encoding a particular gene product.

The term “transcriptional control” is intended to include the ability to modulate gene expression at the level of transcription. In certain embodiments, transcription, and thus gene expression, is modulated by replacing or adding a surrogate promoter near the 5′ end of the coding region of a gene-of-interest, thereby resulting in altered gene expression. In certain embodiments, the transcriptional control of one or more gene is engineered to result in the optimal expression of such genes, e.g., in a desired ratio. The term also includes inducible transcriptional control as recognized in the art.

The term “expression” is intended to include the expression of a gene at least at the level of mRNA production.

The term “expression product” is intended to include the resultant product, e.g., a polypeptide, of an expressed gene.

The term “increased expression” is intended to include an alteration in gene expression at least at the level of increased mRNA production and, preferably, at the level of polypeptide expression. The term “increased production” is intended to include an increase in the amount of a polypeptide expressed, in the level of the enzymatic activity of the polypeptide, or a combination thereof.

The terms “activity,” “activities,” “enzymatic activity,” and “enzymatic activities” are used interchangeably and are intended to include any functional activity normally attributed to a selected polypeptide when produced under favorable conditions. Typically, the activity of a selected polypeptide encompasses the total enzymatic activity associated with the produced polypeptide. The polypeptide produced by a host cell and having enzymatic activity can be located in the intracellular space of the cell, cell-associated, secreted into the extracellular milieu, or a combination thereof. Techniques for determining total activity as compared to secreted activity are described herein and are known in the art.

The term “cellulolytic activity” is intended to include the ability to hydrolyze glycosidic linkages in oligohexoses and polyhexoses. Cellulolytic activity can also include the ability to depolymerize or debranch cellulose and hemicellulose.

As used herein, the term “lactoyl-CoA” is intended to include an intermediate product generated in a metabolic pathway involving the conversion of lactate to acryloyl-CoA.

As used herein, the term “β-alanyl-CoA” is intended to include an intermediate product generated in a metabolic pathway involving the conversion of β-alanine to acryloyl-CoA.

As used herein, the term “acryloyl-CoA” is intended to include an intermediate product generated in a metabolic pathway involving the conversion of lactoyl-CoA to acrylate, or involving the conversion of β-alanyl-CoA to acrylate.

As used herein, the term “lactoyl-CoA dehydratase” is an enzyme that catalyzes the chemical reaction: lactoyl-CoA→acryloyl-CoA+H₂O. Hence, this enzyme has one substrate, lactoyl-CoA, and two products, acryloyl-CoA and H₂O. This enzyme belongs to the family of lyases, specifically the hydro-lyases, which cleave carbon-oxygen bonds. Other names to refer to lactyol-CoA dehdratase include lactoyl coenzyme A dehydratase, lactyl-coenzyme A dehydrase, lactyl CoA dehydratase, acrylyl coenzyme A hydratase, and lactoyl-CoA hydro-lyase. This enzyme participates in propanoate metabolism.

As used herein, the term “alanine aminotransferase” or “ALT” is a transaminase enzyme. It is also called “serum glutamic pyruvic transaminase” (SGPT) or “alanine aminotransferase” (ALAT).

As used herein, the term “glutamate dehydrogenase” is an enzyme that converts glutamate to α-Ketoglutarate, and vice versa.

As used herein, the term “aspartate 4-decarboxylase” refers to an enzyme that catalyzes the chemical reaction: L-aspartate→L-alanine+CO2. Hence, this enzyme has one substrate, L-aspartate, and two products, L-alanine and CO2. This enzyme belongs to the family of lyases, specifically the carboxy-lyases, which cleave carbon-carbon bonds. The systematic name of this enzyme class is L-aspartate 4-carboxy-lyase (L-alanine-forming). Other names in common use include desulfinase, aminomalonic decarboxylase, aspartate beta-decarboxylase, aspartate omega-decarboxylase, aspartic omega-decarboxylase, aspartic beta-decarboxylase, L-aspartate beta-decarboxylase, cysteine sulfinic desulfinase, L-cysteine sulfinate acid desulfinase, and L-aspartate 4-carboxy-lyase. This enzyme participates in alanine and aspartate metabolism.

As used herein, the term “β-alanyl-CoA:ammonia lyase” refers to an enzyme involved in the beta-alanine and propanoate metabolism systems. β-Alanyl-CoA is reversibly produced from acrylyl-CoA by enzyme β-alanyl-CoA ammonia-lyase.

As used herein, the term “lactate dehydrogenase” or “LDH” is intended to include the enzyme capable of converting pyruvate into lactate. It is understood that LDH can also catalyze the oxidation of hydroxybutyrate.

As used herein the term “alcohol dehydrogenase” or “ADH” is intended to include the enzyme capable of converting acetaldehyde into an alcohol, such as ethanol.

As used herein, the term “phosphotransacetylase” or “PTA” is intended to include the enzyme capable of converting Acetyl CoA into acetate.

As used herein, the term “acetate kinase” or “ACK” is intended to include the enzyme capable of converting Acetyl CoA into acetate.

As used herein, the term “pyruvate formate lyase” or “PFL” is intended to include the enzyme capable of converting pyruvate into Acetyl CoA.

The term “pyruvate decarboxylase activity” is intended to include the ability of a polypeptide to enzymatically convert pyruvate into acetaldehyde (e.g., “pyruvate decarboxylase” or “PDC”). Typically, the activity of a selected polypeptide encompasses the total enzymatic activity associated with the produced polypeptide, comprising, e.g., the superior substrate affinity of the enzyme, thermostability, stability at different pHs, or a combination of these attributes.

The term “ethanologenic” is intended to include the ability of a microorganism to produce ethanol from a carbohydrate as a fermentation product. The term is intended to include, but is not limited to, naturally occurring ethanologenic organisms, ethanologenic organisms with naturally occurring or induced mutations, and ethanologenic organisms which have been genetically modified.

The terms “fermenting” and “fermentation” are intended to include the enzymatic process (e.g., cellular or acellular, e.g., a lysate or purified polypeptide mixture) by which ethanol is produced from a carbohydrate, in particular, as a product of fermentation.

The term “secreted” is intended to include the movement of polypeptides to the periplasmic space or extracellular milieu. The term “increased secretion” is intended to include situations in which a given polypeptide is secreted at an increased level (i.e., in excess of the naturally-occurring amount of secretion). In certain embodiments, the term “increased secreted” refers to an increase in secretion of a given polypeptide that is at least about 10% or at least about 100%, 200%, 300%, 400%, 500%, 600%, 700%, 800%, 900%, 1000%, or more, as compared to the naturally-occurring level of secretion.

The term “secretory polypeptide” is intended to include any polypeptide(s), alone or in combination with other polypeptides, that facilitate the transport of another polypeptide from the intracellular space of a cell to the extracellular milieu. In certain embodiments, the secretory polypeptide(s) encompass all the necessary secretory polypeptides sufficient to impart secretory activity to a Gram-negative or Gram-positive host cell. Typically, secretory proteins are encoded in a single region or locus that can be isolated from one host cell and transferred to another host cell using genetic engineering. In certain embodiments, the secretory polypeptide(s) are derived from any bacterial cell having secretory activity. In certain embodiments, the secretory polypeptide(s) are derived from a host cell having Type II secretory activity. In certain embodiments, the host cell is a thermophilic bacterial cell.

The term “derived from” is intended to include the isolation (in whole or in part) of a polynucleotide segment from an indicated source or the purification of a polypeptide from an indicated source. The term is intended to include, for example, direct cloning, PCR amplification, or artificial synthesis from or based on a sequence associated with the indicated polynucleotide source.

By “thermophilic” is meant an organism that thrives at a temperature of about 45° C. or higher.

By “mesophilic” is meant an organism that thrives at a temperature of about 20-45° C.

The term “organic acid” is art-recognized. “Organic acid,” as used herein, also includes certain organic solvents such as ethanol. The term “lactic acid” refers to the organic acid 2-hydroxypropionic acid in either the free acid or salt form. The salt form of lactic acid is referred to as “lactate” regardless of the neutralizing agent, i.e., calcium carbonate or ammonium hydroxide. The term “acetic acid” refers to the organic acid methanecarboxylic acid, also known as ethanoic acid, in either free acid or salt form. The salt form of acetic acid is referred to as “acetate.” The term “acrylic acid” refers to the organic acid also referred to as 2-propenoic acid, prop-2-enoic acid, in either free acid or salt form. The salt form of acetic acid is referred to as “acetate.”

Certain embodiments of the present invention provide for the “insertion,” (e.g., the addition, integration, incorporation, or introduction) of certain genes or particular polynucleotide sequences within thermophilic or mesophilic microorganisms, which insertion of genes or particular polynucleotide sequences can be understood to encompass “genetic modification(s)” or “transformation(s)” such that the resulting strains of said thermophilic or mesophilic microorganisms can be understood to be “genetically modified” or “transformed.” In certain embodiments, strains can be of bacterial, fungal, or yeast origin.

Certain embodiments of the present invention provide for the “inactivation” or “deletion” of certain genes or particular polynucleotide sequences within thermophilic or mesophilic microorganisms, which “inactivation” or “deletion” of genes or particular polynucleotide sequences can be understood to encompass “genetic modification(s)” or “transformation(s)” such that the resulting strains of said thermophilic or mesophilic microorganisms can be understood to be “genetically modified” or “transformed.” In certain embodiments, strains can be of bacterial, fungal, or yeast origin.

The term “CBP organism” is intended to include microorganisms of the invention, e.g., microorganisms that have properties suitable for CBP.

In one aspect of the invention, the genes or particular polynucleotide sequences are inserted to activate the activity for which they encode, such as the expression of an enzyme. In certain embodiments, genes encoding enzymes in the metabolic production of ethanol, e.g., enzymes that metabolize pentose and/or hexose sugars, can be added to a mesophilic or thermophilic organism. In certain embodiments of the invention, the enzyme can confer the ability to metabolize a pentose sugar and be involved, for example, in the D-xylose pathway and/or L-arabinose pathway.

In one aspect of the invention, the genes or particular polynucleotide sequences are partially, substantially, or completely deleted, silenced, inactivated, or down-regulated in order to inactivate the activity for which they encode, such as the expression of an enzyme. Deletions provide maximum stability because there is no opportunity for a reverse mutation to restore function. Alternatively, genes can be partially, substantially, or completely deleted, silenced, inactivated, or down-regulated by insertion of nucleic acid sequences that disrupt the function and/or expression of the gene (e.g., P1 transduction or other methods known in the art). The terms “eliminate,” “elimination,” and “knockout” are used interchangeably with the team “deletion,” “partial deletion,” “substantial deletion,” or “complete deletion.” In certain embodiments, strains of thermophilic or mesophilic microorganisms of interest can be engineered by site directed homologous recombination to knockout the production of organic acids. In still other embodiments, RNAi or antisense DNA (asDNA) can be used to partially, substantially, or completely silence, inactivate, or down-regulate a particular gene of interest.

In certain embodiments, the genes targeted for deletion or inactivation as described herein can be endogenous to the native strain of the microorganism, and can thus be understood to be referred to as “native gene(s)” or “endogenous gene(s).” An organism is in “a native state” if it has not been genetically engineered or otherwise manipulated by the hand of man in a manner that intentionally alters the genetic and/or phenotypic constitution of the organism. For example, wild-type organisms can be considered to be in a native state. In other embodiments, the gene(s) targeted for deletion or inactivation can be non-native to the organism.

Biomass

The terms “lignocellulosic material,” “lignocellulosic substrate,” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants, and sugar-processing residues.

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; and forestry wastes, such as but not limited to recycled wood pulp fiber, sawdust, hardwood (e.g., poplar, oak, maple, birch, willow), softwood, or any combination thereof. Lignocellulosic material can comprise one species of fiber; alternatively, lignocellulosic material can comprise a mixture of fibers that originate from different lignocellulosic materials. Particularly advantageous lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Paper sludge is also a viable feedstock for lactate or acetate production. Paper sludge is solid residue arising from pulping and paper-making, and is typically removed from process wastewater in a primary clarifier. At a disposal cost of $30/wet ton, the cost of sludge disposal equates to $5/ton of paper that is produced for sale. The cost of disposing of wet sludge is a significant incentive to convert the material for other uses, such as conversion to ethanol. Processes provided by the present invention are widely applicable. Moreover, the saccharification and/or fermentation products can be used to produce ethanol or higher value added chemicals, such as organic acids, aromatics, esters, acetone and polymer intermediates.

### Acrylate Metabolism

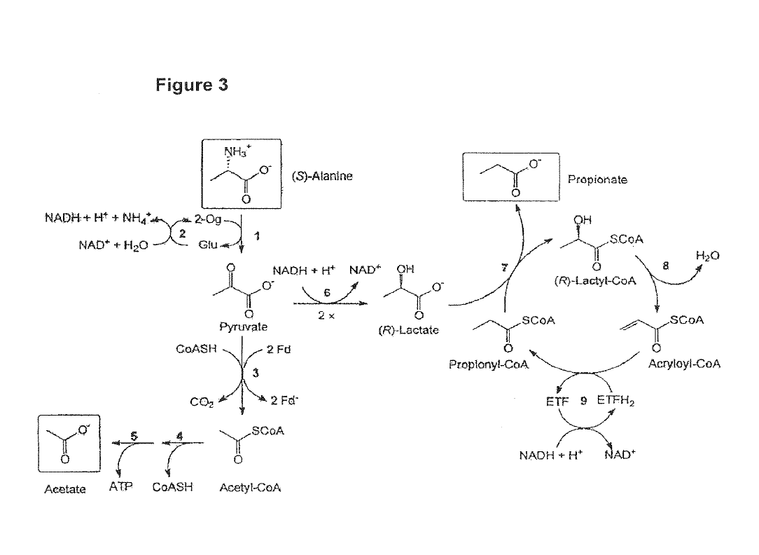

In microbial metabolism, acrylate is rarely seen as an end product, particularly of anaerobic fermentation. More common is the metabolic intermediate acryloyl-CoA, which occurs prominently in the metabolism of the propionic acid producing bacterium Clostridium propionicum and a handful of other prokaryotic species using a similar fermentation pathway. As depicted in the C. propionicum metabolic pathway of FIG. 3, acryloyl-CoA is an intermediate of propionate production. Acrylate is only reported to accumulate as an end product (at most up to 1% of total consumed substrate) when Acrloyl-CoA reductase is blocked by a chemical inhibitor, or by resting cells consuming propionate as an NADH source. This suggests that the native enzymes of C. propionicum have the ability to produce acrylate from acryloyl-CoA or another intermediate, but it is not the primary function of the enzyme(s).

### Lactate

Lactate is produced by NADH-dependent reduction of pyruvate in an enzymatic reaction catalyzed by lactate dehydrogenase (Ldh). Both C. thermocellum and C. cellulolyticum make lactate under standard fermentation conditions and have well annotated genes encoding Ldh (see Table below). Lactate yield can be increased by partial, substantial, or complete deletion, silencing, inactivation, or down-regulation of single genes or combinations of genes in competing pathways leading to acetate, ethanol, and formate production. Key genes to be targeted in these pathways include pta and ack (individual and/or combined mutations) for acetate, adh's for ethanol, and pfl for formate. All of the above genes have been annotated in the published genomes of C. thermocellum and C. cellulolyticum (See Table below). In certain cases (pfl for C. cellulolyticum and adh for both organisms) multiple homologous genes are predicted for a given step.

| | | Published | Published |
|---|---|---|---|
| | | C. thermocellum | C. cellulolyticum |
| Target | gene | 27405 genome | H10 genome |
| Lactate dehydrogenase | ldh | Cthe1053 | Gene 2262 |
| Phosphotransacetylase | pta | Cthe1029 | Gene 132 |
| Acetate Kinase | ack | Cthe1028 | Gene 131 |
| Pyruvate Formate | pfl | Cthe505 | Gene2064 and |
| Lyase | | | Gene 2216 |
| Alcohol | adh | Cthe 423, 394, | Gene 873, 534, |
| dehydrogenase(s) | | 2579, 101, 2238 | 988, 2512 |

### Microorganisms

The present invention includes multiple strategies for the development of microorganisms with the combination of substrate-utilization and product-formation properties required for CBP. The “native cellulolytic strategy” involves engineering naturally occurring cellulolytic microorganisms to improve product-related properties, such as yield and titer. The “recombinant cellulolytic strategy” involves engineering natively non-cellulolytic organisms that exhibit high product yields and titers to express a heterologous cellulase system that enables cellulose utilization or hemicellulose utilization or both.

### Cellulolytic Microorganisms

Several microorganisms reported in the literature to be cellulolytic or have cellulolytic activity have been characterized by a variety of means, including their ability to grow on microcrystalline cellulose as well as a variety of other sugars. Additionally, the organisms can be characterized by other means, including but not limited to, their ability to depolymerize and debranch cellulose and hemicellulose. Celluloytic organisms are described, for example, in U.S. Prov. Appl. No. 61/113,978, the contents of which are herein incorporated by reference.

Certain microorganisms, including, for example, C. thermocellum and C. straminisolvens, cannot metabolize pentose sugars, such as D-xylose or L-arabinose, but are able to metabolize hexose sugars. Both D-xylose and L-arabinose are abundant sugars in biomass with D-xylose accounting for approximately 16-20% in soft and hard woods and L-arabinose accounting for approximately 25% in corn fiber. Accordingly, certain microorganisms of the present invention are genetically-modified to metabolize pentose sugars to enhance their use as biocatalysts for fermentation in the biomass-to-lactic acid industries. Such genetic modifications are also described, for example, in U.S. Prov. Appl. No. 61/113,978.

### Cellulolytic and Xylanolytic Microorganisms

Several microorganisms determined from literature to be both cellulolytic and xylanolytic have been characterized by their ability to grow on microcrystalline cellulose and birchwood xylan as well as a variety of other sugars. Clostridium thermocellum was used to benchmark the organisms of interest, described in U.S. Prov. Appl. No. 61/113,978.

### Transgenic Conversion of Microorganisms

The present invention provides compositions and methods for the transgenic conversion of certain microorganisms. When genes encoding enzymes involved in the metabolic pathway of acrylate, including, for example, lactoyl-CoA, β-alanyl-CoA or acryloyl-CoA, are introduced into a bacterial strain that lacks one or more of these genes, for example, C. thermocellum or T. saccharolyticum, one can select transformed strains for increased production of acrylate. It is expected that genes from other Clostridial species should be expressed in C. thermocellum and T. saccharolyticum. Target gene donors can include microorganisms that confer the ability to convert pyruvate to acrylate, e.g., C. propionicum.

### Transposons

To select for foreign DNA that has entered a host it is preferable that the DNA be stably maintained in the organism of interest. With regard to plasmids, there are two processes by which this can occur. One is through the use of replicative plasmids. These plasmids have origins of replication that are recognized by the host and allow the plasmids to replicate as stable, autonomous, extrachromosomal elements that are partitioned during cell division into daughter cells. The second process occurs through the integration of a plasmid onto the chromosome. This predominately happens by homologous recombination and results in the insertion of the entire plasmid, or parts of the plasmid, into the host chromosome. Thus, the plasmid and selectable marker(s) are replicated as an integral piece of the chromosome and segregated into daughter cells. Therefore, to ascertain if plasmid DNA is entering a cell during a transformation event through the use of selectable markers requires the use of a replicative plasmid or the ability to recombine the plasmid onto the chromosome. These qualifiers cannot always be met, especially when handling organisms that do not have a suite of genetic tools.

One way to avoid issues regarding plasmid-associated markers is through the use of transposons. A transposon is a mobile DNA element, defined by mosaic DNA sequences that are recognized by enzymatic machinery referred to as a transposase. The function of the transposase is to randomly insert the transposon DNA into host or target DNA. A selectable marker can be cloned onto a transposon by standard genetic engineering. The resulting DNA fragment can be coupled to the transposase machinery in an in vitro reaction and the complex can be introduced into target cells by electroporation. Stable insertion of the marker onto the chromosome requires only the function of the transposase machinery and alleviates the need for homologous recombination or replicative plasmids.

### Native Cellulolytic Strategy

Naturally occurring cellulolytic microorganisms are starting points for CBP organism development via the native strategy. Anaerobes and facultative anaerobes are of particular interest. The primary objective is to engineer product yields and acrylate titers to satisfy the requirements of an industrial process. Metabolic engineering of mixed-acid fermentations in relation to, for example, ethanol production, has been successful in the case of mesophilic, non-cellulolytic, enteric bacteria. Recent developments in suitable gene-transfer techniques allow for this type of work to be undertaken with cellulolytic bacteria.

### Recombinant Cellulolytic Strategy

Non-cellulolytic microorganisms with desired product-formation properties (e.g., high acrylate yield and titer) are starting points for CBP organism development by the recombinant cellulolytic strategy. The primary objective of such developments is to engineer a heterologous cellulase system that enables growth and fermentation on pretreated lignocellulose. The heterologous production of cellulases has been pursued primarily with bacterial hosts producing ethanol at high yield (engineered strains of E. coli, Klebsiella oxytoca, and Zymomonas mobilis) and the yeast Saccharomyces cerevisiae. Cellulase expression in strains of K. oxytoca resulted in increased hydrolysis yields—but not growth without added cellulase—for microcrystalline cellulose, and anaerobic growth on amorphous cellulose. Although dozens of saccharolytic enzymes have been functionally expressed in S. cerevisiae, anaerobic growth on cellulose as the result of such expression has not been definitively demonstrated.

Aspects of the present invention relate to the use of thermophilic or mesophilic microorganisms as hosts for modification via the native cellulolytic strategy. Their potential in process applications in biotechnology stems from their ability to grow at relatively high temperatures with attendant high metabolic rates, production of physically and chemically stable enzymes, and elevated yields of end products. Major groups of thermophilic bacteria include eubacteria and archaebacteria. Thermophilic eubacteria include: phototropic bacteria, such as cyanobacteria, purple bacteria, and green bacteria; Gram-positive bacteria, such as Bacillus, Clostridium, Lactic acid bacteria, and Actinomyces; and other eubacteria, such as Thiobacillus, Spirochete, Desulfotomaculum, Gram-negative aerobes, Gram-negative anaerobes, and Thermotoga. Within archaebacteria are considered Methanogens, extreme thermophiles (an art-recognized term), and Thermoplasma. In certain embodiments, the present invention relates to Gram-negative organotrophic thermophiles of the genera Thermus, Gram-positive eubacteria, such as genera Clostridium, and also which comprise both rods and cocci, genera in group of eubacteria, such as Thermosipho and Thermotoga, genera of Archaebacteria, such as Thermococcus, Thermoproteus (rod-shaped), Thermofilum (rod-shaped), Pyrodictium, Acidianus, Sulfolobus, Pyrobaculum, Pyrococcus, Thermodiscus, Staphylothermus, Desulfurococcus, Archaeoglobus, and Methanopyrus. Some examples of thermophilic or mesophilic (including bacteria, procaryotic microorganism, and fungi), which can be suitable for the present invention include, but are not limited to: Clostridium thermosulfurogenes, Clostridium cellulolyticum, Clostridium thermocellum, Clostridium thermohydrosulfuricum, Clostridium thermoaceticum, Clostridium thermosaccharolyticum, Clostridium tartarivorum, Clostridium thermocellulaseum, Clostridium phytofermentans, Clostridium straminosolvens, Thermoanaerobacterium thermosaccarolyticum, Thermoanaerobacterium saccharolyticum, Thermobacteroides acetoethylicus, Thermoanaerobium brockii, Methanobacterium thermoautotrophicum, Anaerocellum thermophilium, Pyrodictium occultum, Thermoproteus neutrophilus, Thermofilum librum, Thermothrix thioparus, Desulfovibrio thermophilus, Thermoplasma acidophilum, Hydrogenomonas thermophilus, Thermomicrobium roseum, Thermus Havas, Thermus ruber, Pyrococcus furiosus, Thermus aquaticus, Thermus thermophilus, Chloroflexus aurantiacus, Thermococcus litoralis, Pyrodictium abyssi, Bacillus stearothermophilus, Cyanidium caldarium, Mastigocladus laminosus, Chlamydothrix calidissima, Chlamydothrix penicillata, Thiothrix carnea, Phormidium tenuissimum, Phormidium geysericola, Phormidium subterraneum, Phormidium bijahensi, Oscillatoria filiformis, Synechococcus lividus, Chloroflexus aurantiacus, Pyrodictium brockii, Thiobacillus thiooxidans, Sulfolobus acidocaldarius, Thiobacillus thermophilica, Bacillus stearothermophilus, Cercosulcifer hamathensis, Vahlkampfia reichi, Cyclidium citrullus, Dactylaria gallopava, Synechococcus lividus, Synechococcus elongatus, Synechococcus minervae, Synechocystis aquatilus, Aphanocapsa thermalis, Oscillatoria terebriformis, Oscillatoria amphibia, Oscillatoria germinata, Oscillatoria okenii, Phormidium laminosum, Phormidium parparasiens, Symploca thermalis, Bacillus acidocaldarias, Bacillus coagulans, Bacillus thermocatenalatus, Bacillus licheniformis, Bacillus pamilas, Bacillus macerans, Bacillus circulars, Bacillus laterosporus, Bacillus brevis, Bacillus subtilis, Bacillus sphaericus, Desulfotomaculum nigrificans, Streptococcus thermophilus, Lactobacillus thermophilus, Lactobacillus bulgaricus, Bifidobacterium thermophilum, Streptomyces fragmentosporus, Streptomyces thermonitrificans, Streptomyces thermovulgaris, Pseudonocardia thermophila, Thermoactinomyces vulgaris, Thermoactinomyces sacchari, Thermoactinomyces candidas, Thermomonospora curvata, Thermomonospora viridis, Thermomonospora citrina, Microbispora thermodiastatica, Microbispora aerata, Microbispora bispora, Actinobifida dichotomica, Actinobifida chromogena, Micropolyspora caesia, Micropolyspora faeni, Micropolyspora cectivugida, Micropolyspora cabrobrunea, Micropolyspora thermovirida, Micropolyspora viridinigra, Methanobacterium thermoautothropicum, Caldicellulosiruptor acetigenus, Caldicellulosiruptor saccharolyticus, Caldicellulosiruptor kristjanssonii, Caldicellulosiruptor owensensis, Caldicellulosiruptor lactoaceticus, variants thereof, and/or progeny thereof.

In particular embodiments, the present invention relates to thermophilic bacteria selected from the group consisting of Clostridium cellulolyticum, Clostridium thermocellum, and Thermoanaerobacterium saccharolyticum.

In certain embodiments, the present invention relates to thermophilic bacteria selected from the group consisting of Fervidobacterium gondwanense, Clostridium thermolacticum, Moorella sp., and Rhodothermus marinus.

In certain embodiments, the present invention relates to thermophilic bacteria of the genera Thermoanaerobacterium or Thermoanaerobacter, including, but not limited to, species selected from the group consisting of: Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brockii, variants thereof, and progeny thereof.

In certain embodiments, the present invention relates to microorganisms of the genera Geobacillus, Saccharococcus, Paenibacillus, Bacillus, and Anoxybacillus, including, but not limited to, species selected from the group consisting of: Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, variants thereof, and progeny thereof.

In certain embodiments, the present invention relates to mesophilic bacteria selected from the group consisting of Saccharophagus degradans; Flavobacterium johnsoniae; Fibrobacter succinogenes; Clostridium hungatei; Clostridium phytofermentans; Clostridium cellulolyticum; Clostridium aldrichii; Clostridium termitididis; Acetivibrio cellulolyticus; Acetivibrio ethanolgignens; Acetivibrio multivorans; Bacteroides cellulosolvens; and Alkalibacter saccharofomentans, variants thereof and progeny thereof.

### Methods of the Invention

During glycolysis, cells convert simple sugars, such as glucose, into pyruvic acid, with a net production of ATP and NADH. In the absence of a functioning electron transport system for oxidative phosphorylation, at least 95% of the pyruvic acid is consumed in short pathways which regenerate NAD⁺, an obligate requirement for continued glycolysis and ATP production. The waste products of these NAD⁺ regeneration systems are commonly referred to as fermentation products.

Microorganisms produce a diverse array of fermentation products, including organic acids, such as lactate (the salt form of lactic acid), acetate (the salt form of acetic acid), succinate, and butyrate, and neutral products, such as ethanol, butanol, acetone, and butanediol. End products of fermentation share to varying degrees several fundamental features, including: they are relatively nontoxic under the conditions in which they are initially produced, but become more toxic upon accumulation; and they are more reduced than pyruvate because their immediate precursors have served as terminal electron acceptors during glycolysis.

Aspects of the present invention relate to the use of a recombinant thermophilic or mesophilic microorganism, where the thermophilic or mesophilic microorganism heterologously expresses one or more enzymes and where the one or more enzymes function in an engineered metabolic pathway to convert glucose to acrylate. In certain embodiments, a recombinant thermophilic or mesophilic microorganism is capable of converting glucose to pyruvate utilizing the expresion of native enzymes. In further embodiments, the one or more heterologously expressed enzymes functions in a step of said pathway to convert pyruvate to acrylate.

In certain aspects of the invention, the engineered metabolic pathway comprises the following steps: (a) conversion of pyruvate to lactate; and (b) direct conversion of lactate to acrylate; where step (b) is performed subsequent to step (a). In other aspects, the engineered metabolic pathway comprises the following steps: (a) conversion of pyruvate to lactate; (b) conversion of lactate to lactoyl-CoA; (c) conversion of lactoyol-CoA to acryloyl-CoA; and (d) conversion of acryloyl-CoA to acrylate; where step (b) is performed subsequent to step (a), step (c) is performed subsequent to step (b); and step (d) is performed subsequent to step (c). In yet other aspects, the engineered metabolic pathway comprises the following steps: (a) conversion of pyruvate to L-α-alanine; (b) conversion of L-α-alanine to L-asparate; (c) conversion of L-asparate to β-alanine; (d) conversion of β-alanine to β-alanyl-CoA; (e) conversion of β-alanyl-CoA to acryloyl-CoA; and (f) conversion of acryloyl-CoA to acrylate; where step (b) is performed subsequent to step (a), step (c) is performed subsequent to step (b); step (d) is performed subsequent to step (c); step (e) is performed subsequent to step (d); and step (f) is performed subsequent to step (e).

In additional embodiments of the invention, the recombinant thermophilic or mesophilic microorganism heterologously expresses one or more enzymes, where the one or more enzymes is selected from the group consisting of the following C. propionicum enzymes: D-lactate dehydrogenase (EC 1.1.1.28), hypothetical lactate dehydratase (EC 4.2.1.x), D-lactoyl-CoA dehydratase (EC 4.2.1.54), hypothetical lactate (acrylate) CoA transferase (EC 2.8.3.x), alanine aminotransferase (EC 2.6.1.2), glutamate dehydrogenase (EC 1.4.1.3), aspartate 4-decarboxylase (EC 4.1.1.11), 3-alanyl-CoA:ammonia lyase (EC 4.3.1.6) and hypothetical β-alanine (acrylate) CoA transferase (EC 2.8.3.x), or selected from the group consisting of an enzyme having at least 85%, 90%, 95%, or 97% identity with an enzyme from Clostridium propionicum as listed above.

Further aspects of the present invention relate to the use of gene knockout technology to provide novel microorganisms useful in the increased production of lactate from lignocellulosic biomass substrates. The transformed organisms are prepared by deleting or inactivating one or more genes that encode competing pathways, such as the non-limiting pathways to organic acids described herein, optionally followed by a growth-based selection for mutants with improved performance for producing lactate as a fermentation product.

In certain embodiments, a thermophilic or mesophilic microorganism, which in a native state contains at least one gene that confers upon the microorganism an ability to produce acetic acid as a fermentation product, is transformed to eliminate expression of said at least one gene. The gene that confers upon the microorganism an ability to produce acetic acid as a fermentation product can code for expression of acetate kinase phosphotransacetylase, pyruvate formate lyase, and/or aldehyde or alcohol dehydrogenase. The deletion or suppression of the gene(s) or particular polynucleotide sequence(s) that encode for expression of ACK, PTA, PFL, and/or ADH diminishes or eliminates the reaction scheme in the overall glycolytic pathway whereby pyruvate is converted to acetyl CoA and acetyl CoA is converted to acetic acid or ethanol; the resulting diversion of pyruvate from these later stages of glycolysis to be converted upstream by lactate dehydrogenase should allow for the increased production of lactate.

In certain embodiments, the above-detailed gene knockout schemes can be applied individually or in concert. Eliminating the mechanism for the production of acetate or ethanol (i.e., knocking out the genes or particular polynucleotide sequences that encode for expression of PFL, PTA, ACK, and/or ADH) can be converted more efficiently by lactate dehydrogenase, which should result in increased production of lactate.

In certain embodiments, it is not required that the thermophilic or mesophilic microorganisms have native or endogenous LDH, ACK, PTA, PFL, PDC or ADH. In certain embodiments, the genes encoding for LDH, ACK, PTA, PFL, PDC and/or ADH can be expressed recombinantly in the genetically modified microorganisms of the present invention. In certain embodiments, the gene knockout technology of the present invention can be applied to recombinant microorganisms, which can comprise a heterologous gene that codes for LDH, ACK, PTA, PFL, PDC and/or ADH, wherein said heterologous gene is expressed at sufficient levels to increase the ability of said recombinant microorganism (which can be thermophilic) to produce lactate as a fermentation product or to confer upon said recombinant microorganism (which can be thermophilic) the ability to produce lactate as a fermentation product.

In certain embodiments, aspects of the present invention relate to fermentation of lignocellulosic substrates to produce acrylate in a concentration that is at least 70% of a theoretical yield based on cellulose content or hemicellulose content or both.

In certain embodiments, aspects of the present invention relate to fermentation of lignocellulosic substrates to produce acrylate in a concentration that is at least 80% of a theoretical yield based on cellulose content or hemicellulose content or both.

In certain embodiments, aspects of the present invention relate to fermentation of lignocellulosic substrates to produce acrylate in a concentration that is at least 90% of a theoretical yield based on cellulose content or hemicellulose content or both.

The ability to redirect electron flow by virtue of modifications to carbon flow has broad implications. For example, this approach could be used to produce high lactate or acrylate yields in strains other than T. saccharolyticum.

Metabolic engineering of knockouts can be achieved by methods known to one of ordinary skill in the art, such as recombinant DNA technology, single or double crossover recombinant technology, or antisense oligonucleotide (asRNA) strategies, as described in U.S. Prov. Appl. No. 61/113,978, the contents of which are herein incorporated by reference.

### Design of Antisense Sequences

asRNAs are typically 18-25 nucleotides in length. There are several computation tools available for rational design of RNA-targeting nucleic acids (Sfold, Integrated DNA Technologies, STZ Nucleic Acid Design) which can be used to select asRNA sequences. For instance, the gene sequence for Clostridium thermocellum ack (acetate kinase) can be submitted to a rational design server and several asRNA sequences can be culled. In brief, the design parameters select for mRNA target sequences that do not contain predicted secondary structure.

### Design of Delivery Vector

A replicative plasmid will be used to deliver the asRNA coding sequence to the target organism. Vectors such as, but not limited to, pNW33N, pJIR418, pJIR751, and pCTC1, will form the backbone of the asRNA constructs for delivery of the asRNA coding sequences to inside the host cell. In addition to extra-chromosomal (plasmid based) expression, asRNAs can be stably inserted at a heterologous locus into the genome of the microorganism to get stable expression of asRNAs. In certain embodiments, strains of thermophilic or mesophilic microorganisms of interest can be engineered by site directed homologous recombination to knockout the production of organic acids and other genes of interest can be partially, substantially, or completely deleted, silenced, inactivated, or down-regulated by asRNA.

### Promoter Choice

To ensure expression of asRNA transcripts, compatible promoters for the given host will be fused to the asRNA coding sequence. The promoter-asRNA cassettes are constructed in a single PCR step. Sense and antisense primers designed to amplify a promoter region will be modified such that the asRNA sequence (culled from the rational design approach) is attached to the 5′ end of the antisense primer. Additionally, restriction sites, such as EcoRI or BamHI, will be added to the terminal ends of each primer so that the final PCR amplicon can be digested directly with restriction enzymes and inserted into the vector backbone through traditional cloning techniques.

With respect to microorganisms that do not have the ability to metabolize pentose sugars, but are able to metabolize hexose sugars as described herein, it will be appreciated that the ack and ldh genes of Clostridium thermocellum and Clostridium straminisolvens, for example, can be targeted for inactivation using antisense RNA according to the methods described herein.

With respect to microorganisms that confer the ability to metabolize pentose and hexose sugars as described herein, it will be appreciated that the ack and ldh genes of Clostridium cellulolyticum, Clostridium phytofermentans and Caldicellulosiruptor kristjanssonii, for example, can be targeted for inactivation using antisense according to the methods described herein.

In addition to antibiotic selection for strains expressing the asRNA delivery vectors, such strains can be selected on conditional media that contains any of the several toxic metabolite analogues such as sodium fluoroacetate (SFA), bromoacetic acid (BAA), chloroacetic acid (CAA), 5-fluoroorotic acid (5-FOA) and chlorolactic acid. Use of chemical mutagens including, but not exclusively, ethane methyl sulfonate (EMS) can be used in combination with the expression of antisense oligonucleotide (asRNA) to generate strains that have one or more genes partially, substantially, or completely deleted, silenced, inactivated, or down-regulated.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Stoichiometric and Thermodynamic Calculations for Acrylate Production

Calculations for the conversion efficiency from raw hardwood feedstock are presented in the Table below. The results demonstrate that theoretically, it is stoichiometrically and thermodynamically possible to produce acrylate from glucose and other sugars, with a mass yield of 0.8 g/g hexose.

The cost of producing acrylate from hardwood feedstock would be relatively inexpensive. The feedstock cost for acrylate or lactate are in the range of $0.05-$0.10 per lb at a feedstock price of $40-$80 per short ton.

Conversion efficiency for raw hardwood feedstock

| | Carbo- | | Conversion efficiencies | | |
|---|---|---|---|---|---|
| | hydrate | Theoretical | Pre- | Fermen- | g Product/ |
| Product | fraction | conversion | treatment | tation | g FS |
| Acrylic | 0.65 | 0.8 | 0.9 | 0.9 | 0.42 |
| Acid | | | | | |
| Lactic | 0.65 | 1.0 | 0.9 | 0.9 | 0.53 |
| Acid | | | | | |
| Ethanol | 0.65 | 0.51 | 0.9 | 0.9 | 0.27 |

Hardwood feedstock cost

| | | | Hardwood feedstock cost | | |
|---|---|---|---|---|---|
| | Feedstock price | Conversion | $/ton | $/lb | $/gal |
| Product | $/dry short ton | efficiency | product | product | product |
| Acrylic | 60 | 0.42 | $142 | $0.071 | 0.57 |
| Acid | | | | | |
| Lactic | 60 | 0.53 | $114 | $0.057 | 0.40 |
| Acid | | | | | |
| Ethanol | 60 | 0.27 | $223 | $0.112 | 1.18 |

Theoretical equations are included below for the simplest pathway to acrylate via lactate. Producing lactate at high yield in either the L- or D-isomer occurs in native organisms and has been engineered in many organisms as well. It is anticipated that producing L- or D-lactate would have a high probability of success in the T. saccharolyticum/C. thermocellum platform.

Notably, acrylate production via the lactate pathway has a small negative ΔG°′ between lactate and acrylate, and as a result there is a theoretical molar ratio of 7.8 to 1 moles acrylate to lactate that can be produced at 55° C. The similar pKa's of acrylate and lactate (4.26 and 3.86, respectively) mean that the intracellular and extracellular ratios of both acids will remain comparable unless there is preferential active transport of one species. This implies that the existence of a cellular membrane and ApH will not influence the molar ratio of acrylate to lactate that can be produced. A similar limitation would likely occur if 3-hydroxypropanoate (pKa 4.5), an isomer of lactate, is used as a metabolic intermediate.

Theoretical equation for acrylate production.

½ glucose+1 ADP+1 Pᵢ→1 acrylate+1 H₂O+1 ATP

Partial equations and Gibbs free energy for simplest pathway to acrylate.

| | | kJ/mol |
|---|---|---|
| a) | ½ glucose + 1 NAD + 1 ADP + 1 Pᵢ → | ΔG°′-56.0 |
| | 1 pyruvate + 1 NADH + 1 ATP | |
| b) | 1 pyruvate + 1 NADH → 1 lactate + 1 NAD | ΔG°′-43.1 |
| c) | 1 lactate → 1 acrylate + 1 H₂O | ΔG°′-5.6 |

By setting ΔG°=0, the ratio of [Acrylate]/[Lactate] can be found at equilibrium conditions. (R=0.008344 kJ/mol*K, T=328 K)

$$Δ {G}^{0} = Δ {G}^{0 ′} + RT ln \frac{[ Acrylate ]}{[ Lactate ]}$$

### Example 2

### Pathways to Acrylate

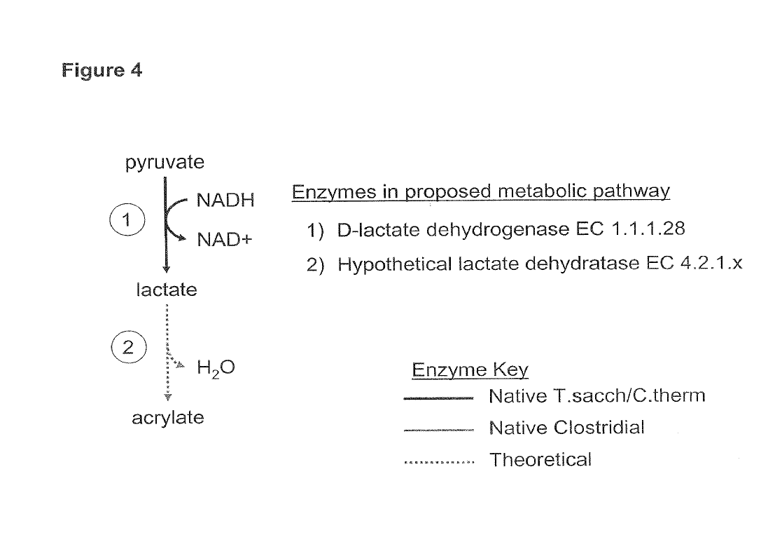

Theoretical pathways to acrylate that have been reported in the literature include ones whose key intermediates include lactoyl-CoA, β-alanyl-CoA, methionine, and 3-hydroxypropanoate. Shown in FIG. 4 are two of the more plausible pathways via lactoyl-CoA and β-alanyl-CoA, as well as a simplified pathway directly from lactate. The other pathways are unlikely to generate net ATP. The pathways presented in FIG. 4 will require enzymes whose activity either has not been reported in the literature, or is not specific to the required substrate. Not having known enzymes available to catalyze necessary steps will require protein engineering or enzyme discovery to create a functioning pathway. Importantly, the final step in all of these pathways leading acrylate does not involve the transfer of electrons or generation of ATP. From a strategic standpoint, this means that cell assisted evolution, such as that done in a cytostat, chemostat, or auxostat will not select for improved acrylate production. This is in contrast to lactate or ethanol, whose production is selected for by more efficient growth.

### Example 3

### Acrylate Production in T. saccharolyticum and C. thermocellum

Both T. saccharolyticum and C. thermocellum natively produce L-lactate, which combined with the many examples of homolactate fermentations suggest that such an engineering project is feasible. D-lactate dehydrogenase could very likely be exchanged with L-lactate dehydrogenase to produce D-lactate. In addition, the flux to ethanol can be limited or eliminated via down-transcription of acetaldehyde and alcohol dehydrogenase.

Examples of strains with increased lactic acid yield are presented below. The results indicated are fermentation data for strains of T. saccharolyticum carrying deletions in the pta/ack, L-ldh, or combined genes. Cultures were grown in batch fermentation at 55° C.

| | | | | | | | | % change in |
|---|---|---|---|---|---|---|---|---|
| | | | | | Yield g/g carbohydrate | | | Y_{LA} |
| Units in grams | | Lactic | Acetic | | consumed | | | relative to |
| per liter | Cellobiose | Acid | Acid | Ethanol | Y_{LA} | Y_{AA} | YEtoh | wildtype |
| M122C | 4.5 | 0 | 0 | 0 | | | | |
| Medium | | | | | | | | |
| M0010 | 0 | 0.77 | 1.04 | 1.4 | 0.17 | 0.23 | 0.31 | 0% |
| wildtype | | | | | | | | |
| M0350 | 2.51 | 0.75 | 0.06 | 0.62 | 0.38 | 0.03 | 0.31 | 120% |
| pta/ack- | | | | | | | | |
| M0350 + pMU42 | 0 | 0 | 0.92 | 1.73 | 0.00 | 0.20 | 0.38 | −100% |
| 4 L-Idh- | | | | | | | | |
| M0353 | 2.15 | 0.02 | 0.09 | 0.95 | 0.01 | 0.04 | 0.40 | −95% |
| pta/ack-, L- | | | | | | | | |
| Idh- | | | | | | | | |

T. saccharolyticum YS485 appears to have many of the enzymes required to produce propionate through the “randomizing” pathway utilized by Propionibacterium species. This pathway produces propionate via the intermediate methylmalonly-CoA rather than acryloyl-CoA, and so is of little value for producing acrylate. Although it is stoichiometrically possible to produce acryloyl-CoA via propionyl-CoA, which is a part of the native T. saccharolyticum pathway, the ΔG°′ of producing acrylate from propionate is +74.1 kJ/mol, making this conversion thermodynamically unfeasible. If ATP were consumed to drive the reaction, there would be no net gain of ATP from the pathway, which would require significant yield reductions due to acetate production for viable cells. C. thermocellum does not have the “randomizing” pathway leading to propionate.

### Example 4

### Knockout Constructs to Increase Production of Lactate

The present invention provides compositions and methods for genetically engineering an organism of interest to CBP by mutating genes encoding key enzymes of metabolic pathways which divert carbon flow away from ethanol and either lactate or acetate towards either lactate or acetate. Single crossover knockout constructs are designed so as to insert large fragments of foreign DNA into the gene of interest to partially, substantially, or completely delete, silence, inactivate, or down-regulate it. Double crossover knockout constructs are designed so as to partially, substantially, or completely delete, silence, inactivate, or down-regulate the gene of interest from the chromosome or replace the gene of interest on the chromosome with a mutated copy of the gene, such as a form of the gene interrupted by an antibiotic resistance cassette.

The design of single crossover knockout vectors requires the cloning of an internal fragment of the gene of interest into a plasmid based system. Ideally, this vector will carry a selectable marker that is expressed in the host strain but will not replicate in the host strain. Thus, upon introduction into the host strain the plasmid will not replicate. If the cells are placed in a conditional medium that selects for the marker carried on the plasmid, only those cells that have found a way to maintain the plasmid will grow. Because the plasmid is unable to replicate as an autonomous DNA element, the most likely way that the plasmid will be maintained is through recombination onto the host chromosome. The most likely place for the recombination to occur is at a region of homology between the plasmid and the host chromosome.

Alternatively, replicating plasmids can be used to create single crossover interruptions. Cells that have taken up the knockout vector can be selected on a conditional medium, then passaged in the absence of selection. Without the positive selection provided by the conditional medium, many organisms will lose the plasmid. In the event that the plasmid is inserted onto the host chromosome, it will not be lost in the absence of selection. The cells can then be returned to a conditional medium and only those that have retained the marker, through chromosomal integration, will grow. A PCR based method will be devised to screen for organisms that contain the marker located on the chromosome.

The design of double crossover knockout vectors requires at least cloning the DNA flanking (˜1 kb) the gene of interest into a plasmid and in some cases can include cloning the gene of interest. A selectable marker can be placed between the flanking DNA or if the gene of interest is cloned the marker is placed internally with respect to the gene. Ideally the plasmid used is not capable of replicating in the host strain. Upon the introduction of the plasmid into the host and selection on a medium conditional to the marker, only cells that have recombined the homologous DNA onto the chromosome will grow. Two recombination events are needed to replace the gene of interest with the selectable marker.

Alternatively, replicating plasmids can be used to create double crossover gene replacements. Cells that have taken up the knockout vector can be selected on a conditional medium, then passaged in the absence of selection. Without the positive selection provided by the conditional medium, many organisms will lose the plasmid. In the event that the drug marker is inserted onto the host chromosome, it will not be lost in the absence of selection. The cells can then be returned to a conditional medium and only those that have retained the marker, through chromosomal integration, will grow. A PCR based method can be devised to screen for organisms that contain the marker located on the chromosome.

In addition to antibiotic selection schemes, several toxic metabolite analogues such as sodium fluoroacetate (SFA), bromoacetic acid (BAA), chloroacetic acid (CAA), 5-fluoroorotic acid (5-FOA) and chlorolactic acid can be used to select mutants arising from either homologous recombinations, or transposon-based strategies. Use of chemical mutagens including, but not exclusively, ethane methyl sulfonate (EMS) can be used in combination with the directed mutagenesis schemes that employ homologous recombinations, or transposon-based strategies.

Exemplary constructs are those as presented in U.S. Prov. Appl. No. 61/113,978, which is herein incorporated by reference.

### Example 5

### Transformation of T. saccharolyticum and C. thermocellum

Cells are grown in 50 mL of GS media with 4 g/l cellobiose to an OD of 0.8 in anaerobic conditions, incubated at 34 degrees C. After harvesting they are washed 3 times in equal volumes with a wash buffer containing 500 mM sucrose and 5 mM MOPS with pH adjusted to 7. After the final wash, the cell pellet is resuspended in an equal volume of wash buffer 10 μl aliquots of the cell suspension are placed in a standard electroporation cuvette with a 1 mm electrode spacing. 1 μl plasmid DNA is added. The concentration of the plasmid DNA is adjusted to ensure between a 1:1 and 10:1 molar ratio of plasmid to cells. A 5 ms pulse is applied with a field strength of 7 kV/cm (measured) across the sample. A custom pulse generator is used. The sample is immediately diluted 1000:1 with the same media used in the initial culturing and allowed to recover until growth resumes, and is determined to increase in the OD (24-48 h). The recovered sample is diluted 50:1 and placed in selective media with either 15 ug/mL erythromycin or 15 ug/mL chloramphenicol and allowed to grow for 5-6 days. Samples exhibiting growth in selective media are tested to confirm that they are in fact T. saccharolyticum or C. thermocellum and that they harbor the plasmid of interest.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A recombinant thermophilic or mesophilic microorganism;
wherein said thermophilic or mesophilic microorganism heterologously expresses one or more enzymes; and
wherein said one or more enzymes function in an engineered metabolic pathway to convert glucose to acrylate.

2. The recombinant thermophilic or mesophilic microorganism of claim 1, wherein said one or more enzyme functions in a step of said pathway to convert pyruvate to acrylate.

3. The recombinant thermophilic or mesophilic microorganism of claim 1, wherein said engineered metabolic pathway comprises the following steps in order: (a) conversion of pyruvate to lactate; and (b) direct conversion of lactate to acrylate.

4. The recombinant thermophilic or mesophilic microorganism of claim 1, wherein said engineered metabolic pathway comprises the following steps in order: (a) conversion of pyruvate to lactate; (b) conversion of lactate to lactoyl-CoA; (c) conversion of lactoyol-CoA to acryloyl-CoA; and (d) conversion of acryloyl-CoA to acrylate.

5. The recombinant thermophilic or mesophilic microorganism of claim 1, wherein said engineered metabolic pathway comprises the following steps in order: (a) conversion of pyruvate to L-α-alanine; (b) conversion of L-α-alanine to L-asparate; (c) conversion of L-asparate to β-alanine; (d) conversion of β-alanine to β-alanyl-CoA; (e) conversion of β-alanyl-CoA to acryloyl-CoA; and (f) conversion of acryloyl-CoA to acrylate.

6. (canceled)

7. The recombinant thermophilic or mesophilic microorganism of claim 3, wherein said one or more enzymes comprises an amino acid sequence having at least 85% identity with the amino acid sequence of an enzyme of Clostridium propionicum that converts pyruvate to lactate or converts lactate to acrylate.

8-10. (canceled)

11. The recombinant thermophilic or mesophilic microorganism of claim 4, wherein said one or more enzymes comprises an amino acid sequence having at least 85% identity with the amino acid sequence of an enzyme of Clostridium propionicum that converts lactoyl-CoA to acryloyl-CoA or converts acryloyl-CoA to acrylate.

12-14. (canceled)

15. The recombinant thermophilic or mesophilic microorganism of claim 5, wherein said one or more enzymes comprises an amino acid sequence having at least 85% identity with the amino acid sequence of an enzyme of Clostridium propionicum that converts converts pyruvate to L-α-alanine, converts L-α-alanine to L-aspartate, converts L-aspartate to β-alanine, converts β-alanyl-CoA to acryloyl-CoA, or converts acryloyl-CoA to acrylate.

16-17. (canceled)

18. The recombinant thermophilic or mesophilic microorganism of claim 1, wherein a first native gene is partially, substantially, or completely deleted, silenced, inactivated, or down-regulated, which first native gene encodes a first native enzyme involved in the metabolic production of an organic acid or a salt thereof, thereby increasing the native ability of said thermophilic or mesophilic microorganism to produce lactate as a fermentation product.

19. The recombinant thermophilic or mesophilic microorganism of claim 18, wherein said first native enzyme is selected from the group consisting of lactate dehydrogenase, acetate kinase, phosphotransacetylase, pyruvate formate lyase, alcohol dehydrogenase, and aldehyde dehydrogenase.

20-25. (canceled)

26. The recombinant thermophilic or mesophilic microorganism of claim 1, wherein said microorganism is a Gram-negative bacterium or a Gram-positive bacterium.

27. The recombinant thermophilic or mesophilic microorganism of claim 26, wherein said microorganism is a species of the genera Thermoanaerobacterium, Thermoanaerobacter, Clostridium, Geobacillus, Saccharococcus, Paenibacillus, Bacillus, Caldicellulosiruptor, Anaerocellum, or Anoxybacillus.

28-29. (canceled)

30. A genetic construct comprising a nucleotide sequence operably linked to a promoter expressible in a thermophilic or mesophilic bacterium, wherein said nucleotide sequence encodes an enzyme of Clostridium propionicum that converts pyruvate to lactate, converts lactate to acrylate, lactoyl-CoA to acryloyl-CoA, converts acryloyl-CoA to acrylate, converts pyruvate to L-α-alanine, converts L-α-alanine to L-aspartate, converts L-aspartate to β-alanine, converts β-alanyl-CoA to acryloyl-CoA, or converts acryloyl-CoA to acrylate, or combinations thereof.

31. (canceled)

32. The genetic construct of claim 30, wherein said nucleotide sequence encodes an enzyme comprising an amino acid sequence having at least 90% identity with the amino acid sequence of an enzyme of Clostridium propionicum that converts pyruvate to lactate or converts lactate to acrylate.

33-34. (canceled)

35. The genetic construct of claim 30, wherein said nucleotide Sequence encodes an enzyme having at least 90% identity with an enzyme of Clostridium propionicum that converts lactoyl-CoA to acryloyl-CoA or converts acryloyl-CoA to acrylate.

36-37. (canceled)

38. The genetic construct of claim 30, wherein said nucleotide sequence encodes an enzyme comprising an amino acid sequence having at least 90% identity with the amino acid sequence of an enzyme of Clostridium propionicum that converts pyruvate to Lα-alanine, converts L-α-alanine to L-aspartate, converts L-aspartate to β-alanine, converts β-alanyl-CoA to acryloyl-CoA, or converts acryloyl-CoA to acrylate.

39. (canceled)

40. A recombinant thermophilic or mesophilic bacterium comprising the genetic construct of claim 30.

41. A vector comprising the genetic construct of any of claim 30.

42. A host cell comprising the genetic construct of claim 30 or the vector of claim 41.

43. The host cell of claim 42, wherein the host cell is a thermophilic or mesophilic bacterial cell.

44. A process for converting lignocellulosic biomass to acrylate, comprising contacting lignocellulosic biomass with a recombinant or genetically modified thermophilic or mesophilic microorganism according to claim 1.

45. The process of claim 44, wherein said lignocellulosic biomass is selected from the group consisting of grass, switch grass, cord grass, rye grass, reed canary grass, mixed prairie grass, miscanthus, sugar-processing residues, sugarcane bagasse, sugarcane straw, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, and combinations thereof.

46. The process of claim 44, wherein said lignocellulosic biomass is selected from the group consisting of corn stover, sugarcane bagasse, switchgrass, and poplar wood.

47-51. (canceled)

52. An engineered process for converting lignocellulosic biomass to acrylate comprising:
contacting said lignocellulosic biomass with a recombinant mesophilic or thermophilic organism, wherein said recombinant mesophilic or thermophilic microorganism converts lignocellulosic biomass to pyruvate, and pyruvate is converted to acrylate using the following steps in order:
(a) conversion of pyruvate to lactate; and
(b) direct conversion of lactate to acrylate.

53. An engineered process for converting lignocellulosic biomass to acrylate comprising:
contacting said lignocellulosic biomass with a recombinant mesophilic or thermophilic organism, wherein said recombinant mesophilic thermophilic microorganism converts lignocellulosic biomass to pyruvate, and pyruvate is converted to acrylate using the following steps in order:
(a) conversion of pyruvate to lactate;
(b) conversion of lactate to lactoyl-CoA;
(c) conversion of lactoyol-CoA to acryloyl-CoA; and
(d) conversion of acryloyl-CoA to acrylate.

54. An engineered process for converting lignocellulosic biomass to acrylate comprising:
contacting said lignocellulosic biomass with a recombinant mesophilic or thermophilic organism, wherein said recombinant thermophilic microorganism converts lignocellulosic biomass to pyruvate, and pyruvate is converted to acrylate using the following steps in order:
(a) conversion of pyruvate to L-α-alanine;
(b) conversion of L-α-alanine to L-asparate;
(c) conversion of L-asparate to β-alanine;
(d) conversion of β-alanine to β-alanyl-CoA;
(e) conversion of β-alanyl-CoA to acryloyl-CoA; and
(f) conversion of acryloyl-CoA to acrylate.

55. (canceled)

56. The engineered process of any one of claims 52-54, wherein said conversion of lignocellulosic biomass to acrylate results in the generation of net positive amount of ATP.

57. The recombinant thermophilic or mesophilic microorganism of claim 7, or the genetic construct of claim 32, wherein said enzyme of Clostridium propionicum that converts pyruvate to lactate or converts lactate to acrylate is selected from the group consisting of D-lactate dehydrogenase and hypothetical lactate dehydratase.

58. The recombinant thermophilic or mesophilic microorganism of claim 11, or the genetic construct of claim 35, wherein said enzyme of Clostridium propionicum that converts lactoyl-CoA to acryloyl-CoA or converts acryloyl-CoA to acrylate is selected from the group consisting of D-lactoyl-CoA dehydratase and hypothetical lactate (acrylate) CoA transferase.

59. The recombinant thermophilic or mesophilic Microorganism of claim 15, or the genetic construct of claim 38, wherein said enzyme of Clostridium propionicum that converts pyruvate to L-α-alanine, converts L-α-alanine to L-αspartate, converts L-aspartate to β-alanine, converts β-alanyl-CoA to acryloyl-CoA, or converts acryloyl-CoA to acrylate is selected from the group consisting of alanine aminotransferase, glutamate dehydrogenase, aspartate 4-decarboxylase, β-alanyl-CoA:ammonia lyase and hypothetical β-alanine (acrylate) CoA transferase.


SEQUENCE LISTING (3 sequences)

SEQ ID NO: 1 - protein, 145 aa, C. propionicum
MVGKKVVHHLMMSAKDAHYTGNLVNGARIVNQWGDVGTELMVYVDGDISLFLGYKDIEFTAPVYVGDFMEYHGWIEKVGNQSYTCKFEAWKVATMVDITNPQDTRATACEPPVLCGRATGSLFIAKKDQRGPQESSFKERKHPGE

SEQ ID NO: 2 - protein, 144 aa, C. proprionicum
MVGKKVVHHLMMSAKDAHYTGNLVNGARIVNQWGDVGTELMVYVDGDISLFLGYKDIEFTAPVYVGDFMEYHGWIEKVGNQSYTCKFEAWKVAKMVDITNPQDTRATACEPPVLCGTATGSLFIAKDNQRGPQESSFKDAKHPQ

SEQ ID NO: 3 - protein, 124 aa, C. propionicum
EFKIAIVDDDLAQESRQIRVDVLDGEGGPLYRMAKAWQQMYGCSLATDTKKGRGRMLINKTIQTGADAIVVAMMKFCDPEEWDYPVMYREFEEKGVKSLMIEVDQEVSSFEQIKTRLQSFVEML